(19) United States
(12) Patent Application Publication
(10) Pub. No.: US 2024/0033102 A1
(43) Pub. Date: Feb. 1, 2024

(54) SURFACE-TREATED INTERVERTEBRAL FUSION CAGE

(71) Applicants: Ming Chi University of Technology, New Taipei City (TW); Chang Gung Memorial Hospital, Linkou, Taoyuan City (TW)

(72) Inventors: PIN-YI CHEN, New Taipei City (TW); PO-LIANG LAI, Taoyuan City (TW); KUEI-CHIH FENG, New Taipei City (TW); CHI-SHUN TU, New Taipei City (TW); CHI-YUN WANG, New Taipei City (TW); YU-JIE WU, New Taipei City (TW); SHYANG-YIH KUNG, New Taipei City (TW); GUAN-YI HUNG, New Taipei City (TW)

(21) Appl. No.: 18/103,839

(22) Filed: Jan. 31, 2023

(30) Foreign Application Priority Data
Jul. 27, 2022 (TW) 111128213

(51) Int. Cl. A61F 2/44; A61F 2/30; C23C 4/02; C23C 4/129; C23C 4/08

(52) U.S. Cl. CPC A61F 2/447; A61F 2/30771; A61F 2/3094; C23C 4/02; C23C 4/129; C23C 4/08; A61F 2310/00407; A61F 2310/00928; A61F 2002/30975; A61F 2002/30784; A61F 2002/3093

(57) ABSTRACT

A surface-treated intervertebral fusion cage is provided, including a main body and a porous structure portion. The porous structure portion is arranged at the corresponding position of the upper and lower parts of the main body. The surface-treated intervertebral fusion cage can improve the biocompatibility, and the surface etching and thermal treatment can increase the coating coverage and increase the mechanical toughness of the cage of the present invention.

### CROSS-REFERENCE TO RELATED APPLICATION

This application claims the priority of Taiwanese patent application No. 111128213, filed on Jul. 27, 2022, which is incorporated herewith by reference.

### BACKGROUND OF THE INVENTION

### 1. Field of the Invention

The present invention relates generally to a surface-treated intervertebral fusion cage, and more particularly, to an intervertebral fusion cage with a surface treated to change material properties.

### 2. The Prior Arts

In modern medicine, putting biomedical implants into living organisms is a fairly mature technology to replace the original joints, intervertebral discs or tooth roots . . . etc. Materials-related research has also become one of the current development priorities in the field of biomedicine.

The vertebral bodies of the vertebrae are not directly and tightly connected, and therefore an intervertebral space exists between the vertebral bodies and an intervertebral disc is arranged in the intervertebral space to support, absorb, and slow down the pressure or shock on the vertebral bodies. For some spine-related conditions, such as spondylolisthesis, herniated intervertebral disc (HIVD), or spinal stenosis that compresses nerves, it is usually necessary to remove the entire or part of the intervertebral disc by surgery, and then a structure that can support the upper and lower vertebral bodies is implanted into the intervertebral space after the intervertebral disc resection to avoid the collapse of the intervertebral space after the intervertebral discectomy or resulting in spinal deformation and other adverse consequences. It can be seen that in order to restore the intervertebral space to maintain the height and to push the vertebral body back to a near-normal position, the implant placed in the intervertebral space needs to be properly selected.

Most of the commercial intervertebral fusion cages do not have biological activity, and most of the materials are still biologically inert materials, such as, titanium alloy and PEEK, and cannot form a good biological link.

As such, the inventor came to the present invention after observing the aforementioned issues.

### SUMMARY OF THE INVENTION

In order to achieve the above objective, the present invention provides a surface-treated intervertebral fusion cage, which is applicable to chiropractic treatment, assists bone hyperplasia, and improves biocompatibility. The surface-treated intervertebral fusion cage mainly includes a main body and a porous structure portion.

The surface of the main body is coated with a titanium alloy of bioactive glass by thermal spraying surface coating technology to improve biocompatibility, and to increase the coating coverage and the mechanical toughness of the intervertebral fusion cage of the present invention by surface etching and thermal treatment.

The porous structure portion comprises biological scaffolds with a porous structure.

Preferably, the end of the main body is designed as wedge-shaped.

Preferably, the porous structure portion comprises biological scaffolds with a porous structure, and the diameter of the pores of the porous structure portion is preferably 0.8-1.2 mm.

In order to make those skilled in the art understand the purpose, features and effects of the present invention, the present invention is described in detail as follows by means of the following specific embodiments and in conjunction with the accompanying drawings.

### BRIEF DESCRIPTION OF THE DRAWINGS

FIG. 1 is a schematic view of a surface-treated intervertebral fusion cage according to the present invention;

FIG. 2 is a schematic side view of a surface-treated intervertebral fusion cage according to the present invention;

FIG. 3 is a schematic view of the porous structure of the surface-treated intervertebral fusion cage according to the present invention;

FIG. 4A is a schematic diagram of stress analysis of the prior art;

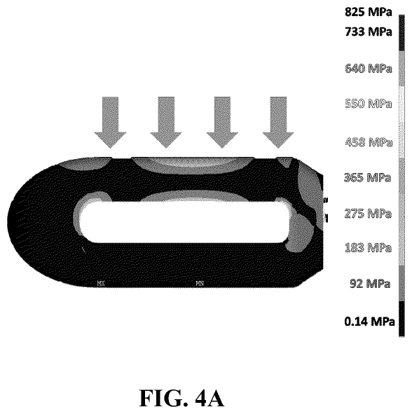

FIG. 4B is a schematic diagram of stress analysis of the surface-treated intervertebral fusion cage according to the present invention;

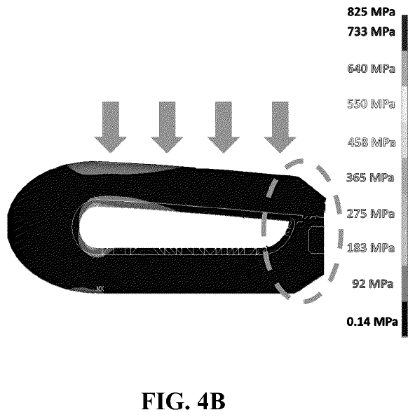

FIG. 5 is a diagram of experimental data of a surface-treated intervertebral fusion cage according to the present invention; and

FIG. 6 is a flow chart of the manufacturing of the surface-treated intervertebral fusion cage according to the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

The terminology used herein is used to describe particular embodiments only, and is not intended to limit the present invention. As used herein, the singular terms “a” and “the” are intended to include the plural forms as well, unless the context clearly dictates otherwise. As used herein, the term “and/or” includes any and all combinations of one or more of the associated listed items. It will be understood that when an element is referred to as being “connected” or “coupled” to another element, it can be directly connected or coupled to the other element or intervening elements may be present.

Exemplary embodiments are described herein with reference to the accompanying drawings, which are schematic illustrations of idealized examples. Accordingly, deviations from the shapes shown, for example, caused by manufacturing techniques and/or tolerances, are expected. Thus, the regions illustrated in the figures are schematic and their shapes are not intended to illustrate the actual shape of a region of a device and are not intended to limit the scope of exemplary embodiments.

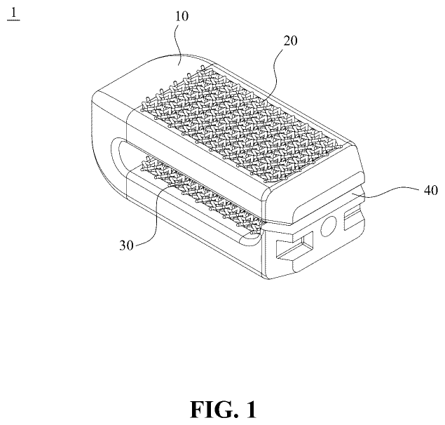

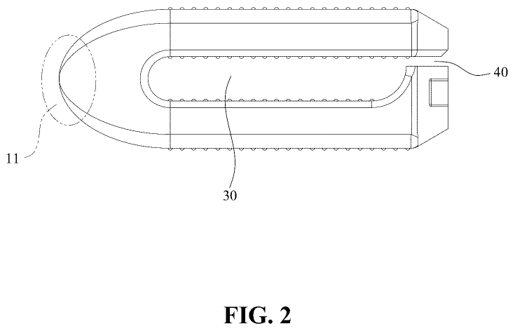

Refer to FIG. 1 and FIG. 2. FIG. 1, which are a schematic view and a side view of the surface-treated intervertebral fusion cage according to the present invention. The surface-treated intervertebral fusion cage 1 mainly includes a main body 10, a porous structure portion 20, a side opening 30, and a rear opening 40; wherein the main body 10 is divided into an upper part and a lower part, and one end of the upper part and one end of the lower part is connected by an end part 11, and the porous structure portion 20 is disposed at the corresponding positions of the upper and lower parts of the main body 10, more precisely, embedded in the corresponding positions of the upper and lower parts of the main body 10.

The side opening 30 is formed by the upper and lower parts of the main body 10 and the end part 11. The rear opening 40 is located at the other end of the upper and lower parts of the main body 10 opposite to the end part 11; that is, one end of the upper part and one end of the lower part of the main body 10 are connected by the end part 11, and the other end is not connected to each other, therefore forming the rear opening 40.

The side opening 30 and the rear opening 40 roughly divide the main body 10 into upper and lower parts, and the upper and lower parts of the main body are connected by the end part 11. The openings are designed to allow flexible intervertebral fusion cage 1 to effectively reduce stress shielding and stress transfer.

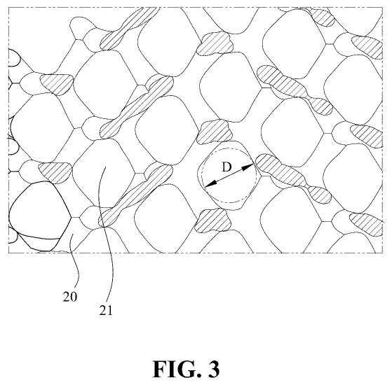

Specifically, refer to FIG. 3. FIG. 3 is a schematic view of the porous structure portion of the surface-treated intervertebral fusion cage according to the present invention. The porous structure portion 20 comprises of biological scaffolds with a porous structure.

Specifically, the biomimetic porous structure of the porous structure portion 20 can promote the attachment and growth of bone cells.

The surface-treated intervertebral fusion cage 1 of the present invention adopts thermal spraying surface coating technology on the surface of the main body to improve biocompatibility, and adopts surface etching and thermal treatment to increase coating coverage and increase the mechanical toughness of the intervertebral fusion cage of the present invention.

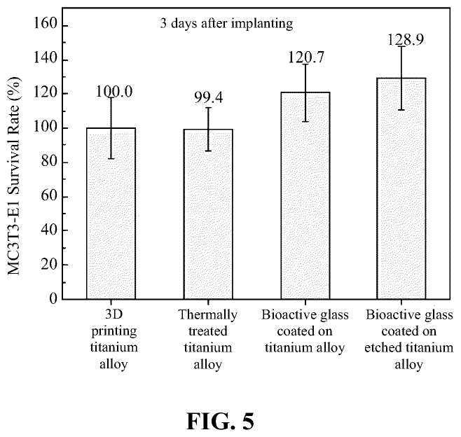

Specifically, the present invention is coated with a titanium alloy with bioactive glass on the surface, and the experimental data of the effect of the surface treatment intervertebral fusion cage 1 on the growth of bone cells after the coating is shown in FIG. 5. The benefits are evident in the data in 3 days after the implanting.

Specifically, as can be seen from FIG. 5, in the osteocyte proliferation experiment, the proliferation rate of the samples with surface treatment is 20% higher than that of the untreated samples. In vitro cell experiments of the surface-treated intervertebral fusion cage, the benefits are more evident in the data in 3 days after the implanting.

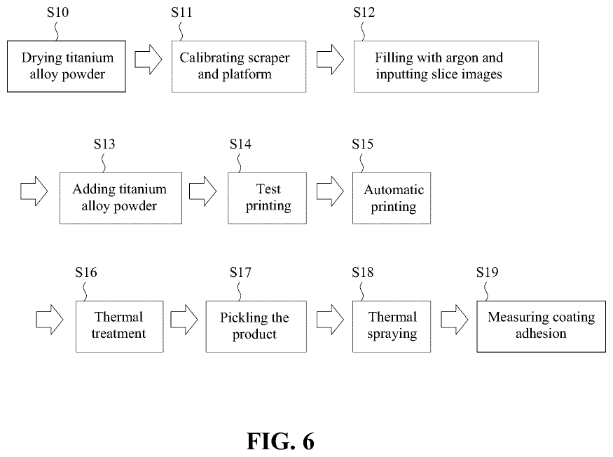

Next, refer to FIG. 6. Steps S10-S15 in FIG. 6 are the process of making the surface-treated intervertebral fusion cage 1.

In step S10, the titanium alloy powder to be printed on the main body 10 is sieved and then dried, so as to increase the fluidity of the powder for spreading the powder more smoothly.

Step S11, calibrating the scraper and the platform, so that the thickness of the powder on a plane is the same.

Step S12, filling with argon to reduce the oxygen content in the cavity to below 1000 ppm to protect the print quality and input slice images.

Step S13, adding titanium alloy powder to the powder spreading chamber in the cavity to facilitate subsequent automatic powder spreading operations.

In steps S14 and S15, automatic printing is performed after test printing; that is, 3D printing of the main body 10 is performed to form a final product.

In step S16, the printed product is subjected to high temperature thermal treatment to remove residual stress generated during the printing process to improve the mechanical toughness of the main body 10.

In step S17, pickling the product after the thermal treatment in step S16 to increase the surface micro-holes, which can increase the adhesion of the coating.

In step S18, the finished product after the pickling is subjected to thermal spray coating of bioactive glass.

In step S19, the coating adhesion is measured according to ASTM C633.

Specifically, the printed product will undergo the process of thermal treatment, pickling, thermal spraying and coating adhesion test to complete the surface-treated intervertebral fusion cage 1 of the present invention.

Specifically, the process steps of thermal spraying, including but not limited to flame spraying, or plasma spraying, comprises: surface roughening treatment, wherein the surface roughness (Ra) must be more than 4 μm; then placing the surface-treated intervertebral fusion cage 1 to be sprayed on the spray fixture and using high-pressure air to cool the back, i.e., the non-spray side, to prevent the surface-treated intervertebral fusion cage 1 from overheating and melting during the spraying process; followed by adjusting the distance between the spray gun and the surface-treated intervertebral fusion cage 1 to 50-100 mm, and setting the parameters of the total number of spraying passes, e.g., 1-6 passes, and the parameters of the moving speed of the spray gun, e.g., 200-450 mm/s; and finally performing thermal spraying after completing the setting.

In summary, the technical features of the present invention and the technical effects that can be achieved are listed as follows:

First, by the surface treatment of the intervertebral fusion cage of the present invention, the surface of the main body uses thermal spraying surface coating technology to improve biocompatibility, and to increase the coating coverage and increase the mechanical properties of the cage of the present invention by surface etching and thermal treatment toughness.

Second, according to the surface-treated intervertebral fusion cage of the present invention, the surface is coated with a titanium alloy of bioactive glass to increase the benefit of bone cell growth.

The above is to illustrate the implementation of the present invention through specific embodiments, those skilled in the art can easily understand other advantages and effects of the present invention from the content disclosed in this specification.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A surface-treated intervertebral fusion cage, comprising:
a main body, divided into an upper part and a lower part, and the upper part and a lower part being connected by an end part;
a porous structure part, disposed at corresponding positions of the upper and lower parts of the main body;
a side opening, formed by the upper part, lower part and the end part of the main body; and
a rear opening, located at the other end of the upper part and lower part opposite to the end part;
wherein one end of the upper part and one end of the lower part being connected by the end part and the other ends of the upper and lower part not connected to each other forming the rear opening;
wherein the side opening extends from the rear opening in a direction towards the end part beyond a boundary of the porous structure portion;
wherein the surface of the main body has a titanium alloy of bioactive glass as a result of both being treated by a thermal spraying surface coating process and thermally treated.

2. The surface-treated intervertebral fusion cage according to claim 1,
wherein the thermal spraying surface coating process comprises the steps of:
roughening surface, wherein the surface roughness (Ra) must be above 4 μm;
placing the surface-treated intervertebral fusion cage to be sprayed on a spraying fixture and using high-pressure air to cool the back side of the surface-treated intervertebral fusion cage to prevent overheating and melting during spraying;
adjusting the distance between a spray gun and surface-treated intervertebral fusion cage, setting parameters for the total number of spraying passes and parameters for the moving speed of the spray gun; and
performing thermal spraying.

3. The surface-treated intervertebral fusion cage according to claim 2, wherein the distance between the spray gun and the surface-treated intervertebral fusion cage is 50-100 mm, the parameter of the total number of spraying passes is 1-6 passes, and the parameter of the moving speed of the spray gun parameter is 200-450 mm/s.
